(19) United States
(12) Patent Application Publication
(10) Pub. No.: US 2025/0262416 A1
(43) Pub. Date: Aug. 21, 2025

(54) GUIDE WIRE AND METHOD FOR PRODUCING GUIDE WIRE

(71) Applicant: ASAHI INTECC CO., LTD., Seto-shi (JP)

(72) Inventors: Yasunao TAKAHASHI, Seto-shi (JP); Rika KUWAHARA, Seto-shi (JP)

(73) Assignee: ASAHI INTECC CO., LTD., Seto-shi (JP)

(21) Appl. No.: 19/196,871

(22) Filed: May 2, 2025

Related U.S. Application Data
(63) Continuation of application No. PCT/JP2023/038280, filed on Oct. 24, 2023.

(30) Foreign Application Priority Data
Nov. 24, 2022 (JP) 2022-187026

(51) Int. Cl. A61M 25/09

(52) U.S. Cl. CPC A61M 25/09; A61M 2025/09083; A61M 2025/09108; A61M 2025/09133; A61M 2205/0216; A61M 2205/0233; A61M 2205/025; A61M 2205/0266; A61M 2207/00

(57) ABSTRACT

A guide wire includes a core wire. The core wire includes a first wire having a proximal end and a second wire having a distal end joined to the proximal end of the first wire. The first wire is formed of a nickel titanium alloy. The second wire is formed of a cobalt chromium alloy. The core wire includes one or more projections projecting from a joint surface between the first wire and the second wire toward a distal end side of the core wire in a longitudinal cross section including the joint surface. The one or more projections are formed of an alloy containing at least one element forming the nickel titanium alloy and at least one element forming the cobalt chromium alloy.

### TECHNICAL FIELD

The technology disclosed herein relates to a guide wire and a method for producing a guide wire.

### BACKGROUND

A technology of forming a core wire by joining two types of wires formed of different materials in order to increase flexibility of a distal end portion of the core wire and increase rigidity of a proximal end portion, in a guide wire to be inserted to a living body lumen such as a blood vessel has been proposed. For example, Patent Literature 1 discloses that, in a guide wire, a core wire is formed by joining a distal end side wire formed of a Ni-Ti based alloy having relatively high flexibility and a proximal end side wire formed of a Co-Cr-Ni based alloy being relatively rigid. Patent Literature 1 discloses that, since, when different metal materials are joined via thermal history in which each of the metal materials are fused, an intermetallic compound is formed in a joint part between the materials and the joint part becomes brittle, it is preferable that a thickness of a joint part between the distal end side wire and the proximal end side wire is equal to or less than 1 μm.

### CITATION LIST

### Patent Literature

### [Patent Literature 1] JP 2017-113267 A

### SUMMARY

### Technical Problem

Patent Literature 1 does not sufficiently consider tensile strength of the core wire forming the guide wire, and there is a room for improvement. An object of the technology disclosed in this specification is to provide a guide wire including a core wire having excellent tensile strength while securing high flexibility in a distal end portion of the core wire and high rigidity in a proximal end portion of the core wire. A technology capable of solving the above problems is disclosed herein.

### Solution to Problem

The technology disclosed herein can be implemented as follows.
(1) A guide wire disclosed in this specification includes a core wire. The core wire includes a first wire having a proximal end and a second wire having a distal end joined to the proximal end of the first wire. The first wire is formed of a nickel titanium alloy. The second wire is formed of a cobalt chromium alloy. The core wire includes one or more projections projecting from a joint surface between the first wire and the second wire toward a distal end side of the core wire in a longitudinal cross section including the joining surface. The one or more projections are formed of an alloy containing at least one element forming the nickel titanium alloy and at least one element forming the cobalt chromium alloy.

### BRIEF DESCRIPTION OF DRAWINGS

FIG. 1 is a diagram that schematically shows the configuration of a guide wire 100 in the embodiment.

FIG. 2 is a diagram that shows the configuration of a joint part X1 between a first wire 11 and a second wire 12.

FIG. 3 is a diagram that shows the configuration of the joint part X1 between the first wire 11 and the second wire 12.

FIG. 4 is a flow chart that shows an example of a method for producing method of the guide wire 100.

FIG. 5 is a diagram that shows examples and a comparative example of a core wire 10 configuring the guide wire 100.

### DETAILED DESCRIPTION OF EMBODIMENTS

### A. Embodiment:

A-1. Configuration of guide wire 100:

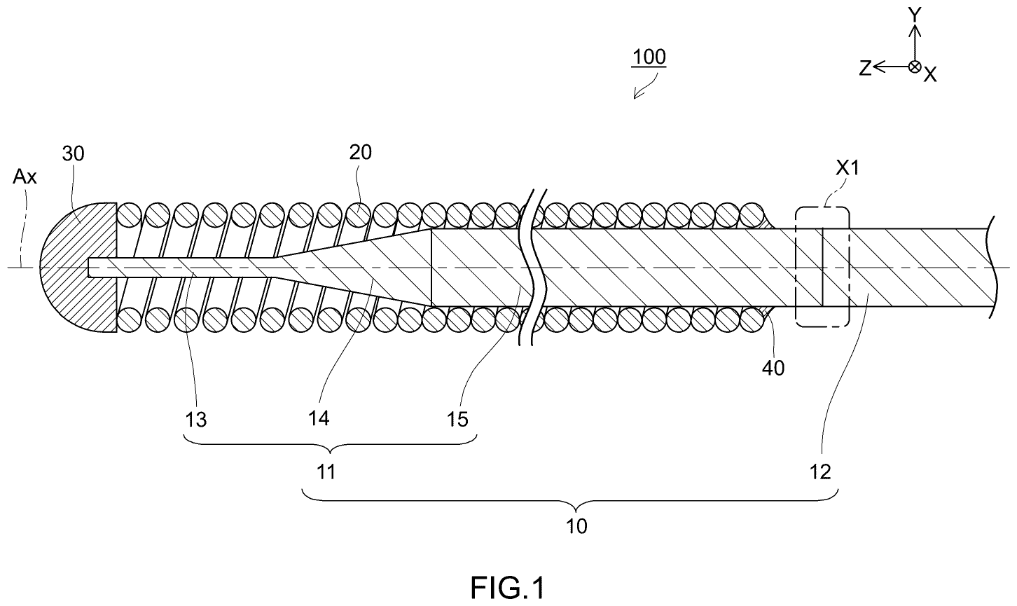

FIG. 1 is a diagram that schematically shows the configuration of the guide wire 100 in the embodiment. FIG. 1 shows a longitudinal cross section (YZ cross section) of the guide wire 100. In FIG. 1, Z axis positive direction side is a distal end side (distal side) to be inserted into a body, and Z axis negative direction side is a proximal end side (near side) to be operated by a professional such as a doctor. In FIG. 1, a portion of the guide wire 100 is omitted. FIG. 1 shows a state where a center axis AX of a core wire 10 described later of the guide wire 100 is a straight line parallel to the Z axis direction, but the guide wire 100 is flexible enough to be bent. The same applies to the following figures.

In the description, regarding the guide wire 100 and the constituent members thereof, an end on the distal end side is referred to as “distal end”, the distal end and a portion in the vicinity thereof are referred to as “distal end portion”, an end on the proximal end side is referred to as “proximal end”, and the proximal end and a portion in the vicinity thereof are referred to as “proximal end portion”. The outer diameters of the guide wire 100 and the constituent members thereof each refer to a size along the direction orthogonal to the center axis AX. Longitudinal cross sections of the guide wire 100 and the constituent members thereof each refer to a cross section including the central axis AX, and transverse sections of the guide wire 100 and the constituent members thereof each refer to a cross section orthogonal to the central axis AX.

The guide wire 100 is a medical device to be inserted to the inside of a living body lumen such as a blood vessel. The guide wire 100 is used for guiding another medical device such as a catheter to a desired position in the inside of the living body lumen. The full length of the guide wire 100 ranges from about 1500 mm to 2000 mm, for example.

The guide wire 100 includes a core wire 10, a coil body 20, a distal end side joint part 30, and a proximal end side joint part 40. At least a part of the guide wire 100 may be coated with, for example, a hydrophilic resin.

The core wire 10 is a long member that extends along the center axis AX and is configured of a metal wire material. The core wire 10 includes a first wire 11 formed of a nickel titanium alloy and a second wire 12 formed of a cobalt chromium alloy. The distal end of the second wire 12 is joined to the proximal end of the first wire 11, for example, by welding. A distance from the distal end of the core wire 10 to the joint part X1 between the first wire 11 and the second wire 12 along the center axis AX is, for example, about 50 mm to 500 mm. In the present embodiment, the joint part X1 between the first wire 11 and the second wire 12 is located at the proximal end side further than the proximal end of the coil body 20. The configuration of the joint part X1 between the first wire 11 and the second wire 12 will be described later in detail.

The nickel titanium alloy being the forming material of the first wire 11 is an alloy containing at least Ni and Ti. The nickel titanium alloy is a flexible material as compared to the cobalt chromium alloy being the forming material of the second wire 12. It is preferable that the nickel titanium alloy contains 40% by mass or more and 60% by mass or less of Ni and contains 40% by mass or more and 60% by mass or less of Ti.

The cobalt chromium alloy being the forming material of the second wire 12 is an alloy containing at least Co and Cr. The cobalt chromium alloy is, for example, a Co-Ni-Cr based alloy, a Co-Cr-Ni-Mo based alloy, a Co-Cr-W-Ni based alloy, a Co-Cr-Mo based alloy, or a Co-Ni-Cr-Mo-W-Fe based alloy. It is preferable that the cobalt chromium alloy is an alloy containing at least Co, Cr, and Ni. For example, it is preferable that the cobalt chromium alloy is an alloy composed of 9 to 40% by mass of Ni, 10 to 30% by mass of Cr, and the remainder being Co, or an alloy having a composition obtained by substituting another element for part of the remainder Co in the above composition. The content of Co may be, for example, 20 to 78% by mass. When the cobalt chromium alloy contains an element other than Co, Cr, and Ni, it is preferable that the total content of the element other than Co, Cr, and Ni is equal to or less than 30% by mass. It is also preferable that the cobalt chromium alloy contains Mo as the element other than Co, Cr, and Ni, and the content of Mo is, for example, 3 to 15% by mass.

As shown in FIG. 1, the first wire 11 includes a small diameter portion 13, a tapered portion 14, and a large diameter portion 15. The small diameter portion 13 is a portion including the distal end of the core wire 10. The large diameter portion 15 is located in a proximal end side with respect to the small diameter portion 13. The outer diameter of the large diameter portion 15 is larger than the outer diameter of the small diameter portion 13 and ranges from about 0.1 mm to 1.0 mm, for example. The tapered portion 14 is located between the small diameter portion 13 and the large diameter portion 15. The outer diameter of the tapered portion 14 gradually increases from the boundary position with respect to the small diameter portion 13, toward the boundary position with respect to the large diameter portion 15. The second wire 12 has a substantially constant outer diameter. The outer diameter of the second wire 12 is substantially equal to the outer diameter of the large diameter portion 15 of the first wire 11. The transverse section at each position of the core wire 10 can have any shape, and is circular or rectangular, for example.

The coil body 20 is a hollow cylindrical coiled member in which one or more wire materials are spirally wound. Each wire material forming the coil body 20 may be configured of a single wire or a twisted wire formed by twisting a plurality of wires. The coil body 20 is arranged so as to surround the outer periphery of the distal end portion of the core wire 10 (specifically, the small diameter portion 13, the tapered portion 14, and a part of the large diameter portion 15 of the first wire 11). The full length of the coil body 20 is about 10 mm to 500 mm, for example, and the outer diameter of the coil body 20 is about 0.2 mm to 1.2 mm, for example.

Examples of materials for forming the coil body 20 include radiolucent materials such as stainless steel (e.g., SUS302, SUS304, and SUS316), a Ni-Ti alloy and a piano wire, and radiopaque materials such as platinum, gold, tungsten or alloys thereof.

The distal end side joint part 30 (distal tip) joins the distal end of the core wire 10 and the distal end of the coil body 20. The outer peripheral surface on the distal end side of the distal end side joint part 30 is a smooth surface (for example, approximate semi-spherical surface). The proximal end side joint part 40 joins the core wire 10 and the proximal end of the coil body 20. Examples of materials for forming the distal end side joint part 30 and the proximal end side joint part 40 include metal solder (e.g., Au-Sn alloy, Sn-Ag alloy, Sn-Pb alloy and Pb-Ag alloy), wax materials (e.g., aluminum alloy solder, silver solder, and gold solder), and adhesives (e.g., epoxy-based adhesive).

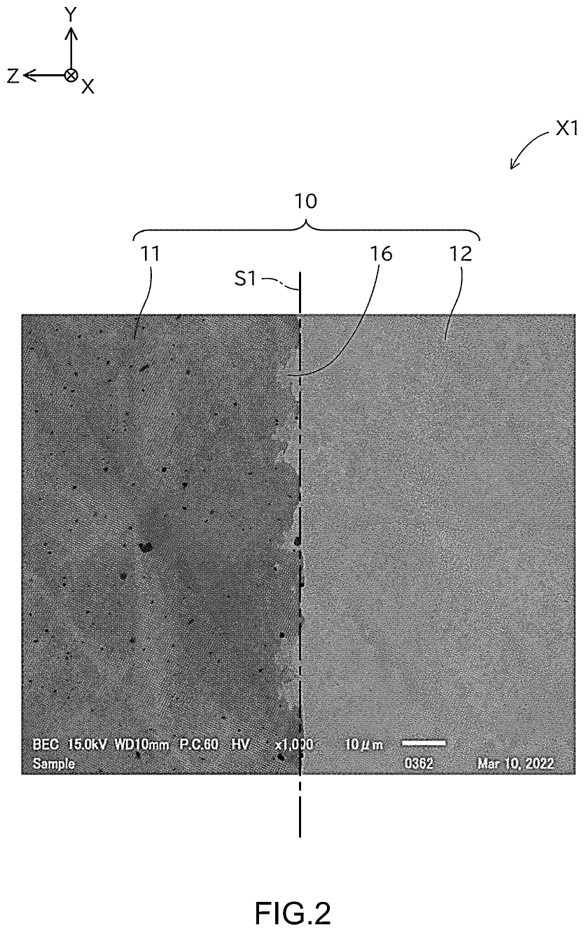

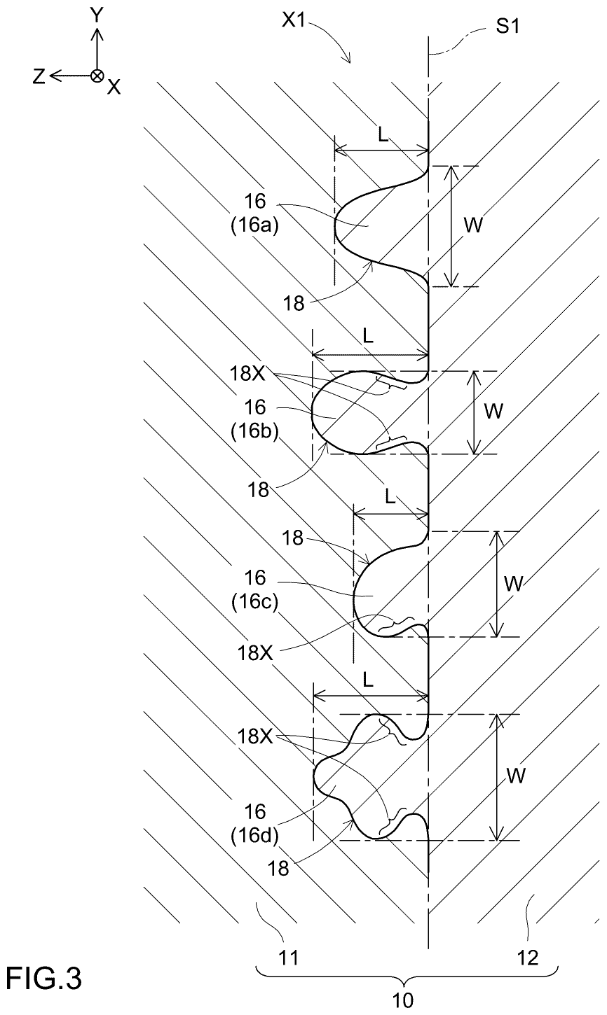

Next, the detailed configuration of the joint part X1 between the first wire 11 and the second wire 12 of the core wire 10 will be described. FIGS. 2 and 3 are diagrams that show the configuration of the joint part X1 between the first wire 11 and the second wire 12. FIG. 2 shows an SEM picture (1000× magnification) of a longitudinal cross section of the joint part X1 between the first wire 11 and the second wire 12, and FIG. 3 schematically shows the configuration of the joint part X1.

As shown in FIGS. 2 and 3, the core wire 10 includes a plurality of projections 16 projecting from a joint surface S1 between the first wire 11 and the second wire 12 toward the distal end side (side of the first wire 11) of the core wire 10.

The projection 16 is formed of an alloy containing at least one element forming a nickel titanium alloy being the forming material of the first wire 11 and at least one element forming a cobalt chromium alloy being the forming material of the second wire 12. It is preferable that the alloy forming the projection 16 is an alloy containing Ni, Ti, Co, and Cr. The alloy forming the projection 16 may be an alloy in which an element forming the nickel titanium alloy is dissolved in a crystal structure of the cobalt chromium alloy, may be an alloy in which, on the contrary, an element forming the cobalt chromium alloy is dissolved in a crystal structure of the nickel titanium alloy, or may be an intermetallic compound formed of the elements forming the nickel titanium alloy and the cobalt chromium alloy. It is preferable that the content ratio of Ni in the alloy forming the projection 16 is higher than the content ratio of Ni in the cobalt chromium alloy. The composition of the alloy forming the projection 16 can be checked by composition analysis using energy dispersive X-ray spectroscopy (EDX).

It is preferable that the alloy forming the projection 16 is a material being more rigid than the nickel titanium alloy and the cobalt chromium alloy. The rigidity of each alloy is measured, for example, by measuring nano indentation rigidity under a condition of the maximum indentation load of 30 mN by using a Berkovich indenter formed of diamond by a nano indenter (iMicro) manufactured by KLA.

It is considered that, since the core wire 10 includes the projection 16 in the joint part X1 between the first wire 11 and the second wire 12, the projection 16 partially intrudes into the nickel titanium alloy side, so that the contact surface with the nickel titanium alloy increases and an anchoring effect occurs, and tensile strength in the joint part X1 between the first wire 11 and the second wire 12 is improved. In view of effectively improving the tensile strength in the joint part X1, it is preferable that the core wire 10 includes the projection 16 having a length L of greater than or equal to 2 μm, it is more preferable that the core wire 10 includes the projection 16 having a length L of greater than or equal to 3 μm, and it is further more preferable that the core wire 10 includes the projection 16 having a length L of greater than or equal to 5 μm. Since the projection 16 may become a start point of breakage, it is preferable that the core wire 10 does not include the projection 16 having a length L exceeding 100 um, and it is more preferable that the core wire 10 does not include the projection 16 having a length L exceeding 50 μm.

The length L of the projection 16 is a length along a direction of the center axis AX of the core wire 10. The length L of the projection 16 is specified as below. As the joint surface S1, an interface is determined where, when surface analysis of a composition using EDX is performed in a longitudinal cross section of the core wire 10, an element (for example, Ti) contained in the nickel titanium alloy is not detected for the first time when proceeding from the first wire 11 toward the proximal end side along the center axis AX. With respect to each projection 16, as a boundary surface between the projection 16 and the nickel titanium alloy, an interface is determined where an element (for example, Co) contained in the cobalt chromium alloy is not detected for the first time when proceeding from the joint surface S1 toward the distal end side along the center axis AX. The length from the joint surface SI to the boundary surface along the center axis AX is the length L of the projection 16.

In this specification, a portion (highly gentle convex portion) having a ratio (W/L) of a width W with respect to the length L is equal to or greater than 3.0 in a convex portion projecting from the joint surface S1 between the first wire 11 and the second wire 12 toward the distal end side does not correspond to the projection 16.

FIG. 3 shows an example of types of shape of the projection 16. In a projection 16a shown in FIG. 3, the contour line 18 does not include a portion extending toward the outer peripheral side of the projection 16 as being closer to the distal end side (left side in the drawing) of the core wire 10. On the other hand, in projections 16b, 16c, and 16d shown in FIG. 3, the contour line 18 includes a portion (hereinafter, referred to as “folding back portion 18X”) extending toward the outer peripheral side of the projection 16 as being closer to the distal end side of the core wire 10. In order to cause a strong anchoring effect by the projection 16, it is preferable that the core wire 10 includes the projection 16 in which the contour line 18 includes the folding back portion 18X.

It is preferable that the core wire 10 includes two or more projections 16 that are substantially not continuous from each other due to the alloy forming the projections 16, in the longitudinal cross section. In this specification, two projections 16 being substantially not continuous from each other due to the alloy means that a thickness of a layer of the alloy existing between the two projections 16 is less than or equal to 1 μm. That is, the configuration where the two projections 16 are substantially not continuous from each other due to the thickness of a layer of the alloy existing between the projections 16 includes a form in which a layer of the alloy exists between the two projections 16 but the thickness of the layer is less than or equal to 1 μm in addition to the form in which a layer of the alloy does not exist. It is preferable that the thickness of the layer of the alloy existing between the two projections 16 is less than or equal to 0.5 μm.

A-2. Method for producing guide wire 100:

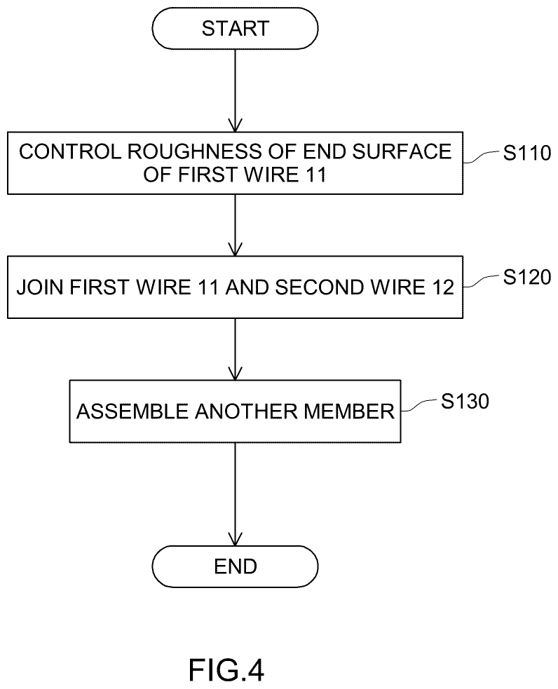

The guide wire 100 of this embodiment can be produced by the following method, for example. FIG. 4 is a flow chart that shows an example of a production method of the guide wire 100.

First, the first wire 11 formed of the nickel titanium alloy is prepared and roughness of an end surface of the proximal end side of the first wire 11 is controlled (S110). More specifically, an arithmetic mean height (Sa) of the end surface on the proximal end side of the first wire 11 is set to be greater than or equal to 0.01 μm and less than or equal to 0.5 μm. The control of the surface roughness can be implemented by, for example, polishing, blasting, plating, etching, electropolishing, thermal spraying. or the like. For example, by polishing using a polishing material, the roughness of the end surface of the first wire 11 can be controlled. At this time, polishing using a polishing material of fine granularity may be followed by polishing using a polishing material of coarse granularity. The control of roughness of the end surface of the first wire 11 may be performed simultaneously with cutting of the first wire 11. For example, by using a cutter capable of simultaneously performing cutting and polishing of a wire, cutting of the first wire 11 and polishing of the end surface may be simultaneously performed.

Next, the core wire 10 is manufactured by joining the distal end of the second wire 12 to the proximal end of the first wire 11 (S120). For example, a right and left pair of clamps grip the first wire 11 and the second wire 12 such that the end surface of the proximal end side of the first wire 11 and the end surface of the distal end side of the second wire 12 face each other. At this time, positions in the first wire 11 and the second wire 12 that are distant from the respective end surfaces by a predetermined distance are gripped by the clamps. By moving the clamps in a direction in which the clamps approach to each other, the end surface of the proximal end side of the first wire 11 and the end surface of the distal end side of the second wire 12 are abutted to each other. By fixing one of the right and left pair of clamps and making the other clamp movable with respect to the fixed clamp, the end surfaces of the first wire 11 and the second wire 12 can be accurately abutted to each other. Next, the first wire 11 and the second wire 12 gripped by the clamps are electrified (heated) while being pressurized. As the electrification condition (current, time, etc.), a condition under which at least one of the end surfaces of the first wire 11 and the second wire 12 is fused is appropriately selected. The entire end surfaces of the first wire 11 and the second wire 12 may be fused or a part of the end surfaces may be fused. By electrifying the first wire 11 and the second wire 12 while pressurizing the first wire 11 and the second wire 12, the end surfaces of both wires are softened and fused, the end surfaces of both wires push each other, so that the end surfaces are deformed so as to spread to the outer peripheral side, and the end surfaces of both wires are joined to each other. As a result of this joining, the projection 16 described above is formed in the joint part X1 between the first wire 11 and the second wire 12.

Thereafter, another member is assembled to the core wire 10 (S130). For example, the coil body 20 is joined to the core wire 10. The guide wire 100 of the embodiment can be produced mainly by the processes described above.

By adjusting the roughness of the end surface of the proximal end side of the first wire 11, the length L of the projection 16 to be formed can be adjusted. For example, when the end surface of the first wire 11 is polished with coarse sandpaper, the length L of the projection 16 tends to be short, and when the end surface of the first wire 11 is polished with fine sandpaper, the length L of the projection 16 tends to be long.

A-3. Effect of the embodiment:

As described above, the guide wire 100 of the embodiment includes the core wire 10. The core wire 10 includes the first wire 11 formed of a nickel titanium alloy and the second wire 12 having a distal end joined to a proximal end of the first wire 11 and being formed of a cobalt chromium alloy. The core wire 10 includes the projection 16 in the longitudinal cross section including the joint surface S1 between the first wire 11 and the second wire 12. The projection 16 is a portion projecting from the joint surface S1 toward the distal end side of the core wire 10, and is formed of an alloy containing at least one element forming a nickel titanium alloy being a forming material of the first wire 11 and at least one element forming a cobalt chromium alloy being a forming material of the second wire 12.

It is conventionally considered that, when different metal materials are joined via thermal history in which each of the metal materials are fused, an intermetallic compound is formed in a joint part between the materials and the joint part becomes brittle and joint strength is reduced. However, the inventors of the present application have conducted intensive studies and found that, when the core wire 10 includes, in the joint part X1 between the first wire 11 and the second wire 12, the projection 16 formed of an alloy containing at least one element forming the nickel titanium alloy being a forming material of the first wire 11 and at least one element forming the cobalt chromium alloy being a forming material of the second wire 12, tensile strength of the joint part X1 is improved. It is considered that this is because the projection 16 partially intrudes into the nickel titanium alloy side, so that the contact area increases and an anchoring effect occurs. Therefore, according to the guide wire 100 of the embodiment, it is possible to provide the guide wire 100 including the core wire 10 having excellent tensile strength while securing high flexibility in a distal end portion (portion formed by the first wire 11) of the core wire 10 and high rigidity in a proximal end portion (portion formed by the second wire 12) of the core wire 10.

In the guide wire 100 of the embodiment, it is preferable that the core wire 10 includes the projection 16 having a length L of equal to or greater than 2 μm along a direction of the center axis AX of the core wire 10 in the longitudinal cross section. With such a configuration, the anchoring effect by the projection 16 can be increased and tensile strength of the core wire 10 can be effectively improved.

In the guide wire 100 of the embodiment, it is preferable that the alloy forming the projection 16 contains Ni, Ti, Co, and Cr. With such a configuration, the joining property of the projection 16 and the nickel titanium alloy and the cobalt chromium alloy can be further improved and tensile strength of the core wire 10 can be further effectively improved.

In the guide wire 100 of the embodiment, it is preferable that the core wire 10 includes the projection 16 such that the projection 16 includes a folding back portion 18X in the longitudinal cross section in which the contour line 18 of the projection 16 extends toward an outer peripheral side of the projection 16 as being closer to the distal end side of the core wire 10. With such a configuration, the anchoring effect by the projection 16 can be further increased and tensile strength of the core wire 10 can be further effectively improved.

In the guide wire 100 of the embodiment, it is preferable that the core wire 10 includes two or more projections 16 that are substantially not continuous from each other due to the thickness of a layer or the alloy existing between the projections 16, in the longitudinal cross section. With such a configuration, due to the existence of the two or more projections 16, tensile strength of the core wire 10 can be further effectively improved.

In the guide wire 100 of the embodiment, the alloy forming the projection 16 is a material being more rigid than either the nickel titanium alloy or the cobalt chromium alloy. With such a configuration, the anchoring effect by the projection 16 being relatively rigid can be increased and tensile strength of the core wire 10 can be further effectively improved.

A method of producing the guide wire 100 of the embodiment includes steps of: controlling a roughness of the end surface of the first wire 11 formed of a nickel titanium alloy such that an arithmetic mean height (Sa) is greater than or equal to 0.01 μm and less than or equal to 0.5 μm; and producing the core wire 10 including the first wire 11 and the second wire 12 by abutting and joining the end surface of the first wire 11 and the end surface of the second wire 12. According to the method of producing the guide wire 100 of the embodiment, the projection 16 can be formed in the joint part X1 between the first wire 11 and the second wire 12, and the guide wire 100 including the core wire 10 having excellent tensile strength can be produced.

### EXAMPLES

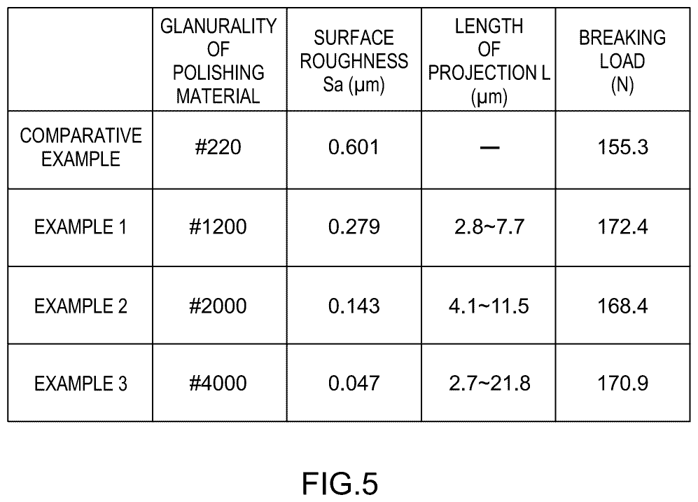

FIG. 5 is a diagram that shows examples and a comparative example of the core wire 10 configuring the guide wire 100. In producing the core wire 10 of the examples and the comparative example, as the first wire 11, a nickel titanium alloy wire (composition (mass %): Ni: 56.1%, C: 0.032%, O: 0.025%, Ti: remainder) having a diameter of 0.42 mm was prepared, and, as the second wire 12, a cobalt chromium alloy wire (composition (mass %): Cr: 20%, Ni: 36.5%, Mo: 10%, Mn: <0.01%, C: 0.002%, Co remainder) having a diameter of 0.42 mm was prepared. By polishing the end surface of the nickel titanium alloy wire by a polishing material having the granularity shown in FIG. 5, the nickel titanium alloy wire having the end surface of the surface roughness (arithmetic mean height Sa) shown in FIG. 5 was obtained. Thereafter, by the method described in the embodiment described above, the first wire 11 and the second wire 12 were joined to produce the core wire 10, and tensile strength of the core wire 10 was evaluated. As the evaluation of the tensile strength of the core wire 10, a sample was set such that the joint part was located at a center part with a chuck interval of 100 mm, and a breakage load when the sample was pulled at a speed of 10 mm/minute was measured. The core wire 10 produced under the same condition was cut in a surface passing the center axis AX of the core wire 10 to obtain a longitudinal cross section, the longitudinal cross section was observed by a scanning electron microscope (SEM), the presence or absence of the projection 16 in the joint part X1 between the first wire 11 and the second wire 12 was checked, and the length L of the projection 16 was measured.

As shown in FIG. 5, in Examples 1 to 3 in which the projection 16 was confirmed in the joint part X1 between the first wire 11 and the second wire 12 of the core wire 10, the breaking load of the core wire 10 was equal to or greater than 168.4 N, which is relatively high. On the other hand, in the comparative example in which the projection 16 was not confirmed in the joint part X1 between the first wire 11 and the second wire 12 of the core wire 10, the breaking load was 155.3 N, which is relatively low. In Examples 1 to 3, it is considered that, the projection 16 formed in the joint part X1 between the first wire 11 and the second wire 12 of the core wire 10 partially intruded into the nickel titanium alloy side, so that the contact surface increased and an anchoring effect occurred, and tensile strength of the core wire 10 was improved.

The disclosed embodiments have an object to improve the tensile strength of a core wire forming a guide wire.

(1) A guide wire disclosed in this specification includes a core wire. The core wire includes a first wire having a proximal end and a second wire having a distal end joined to the proximal end of the first wire. The first wire is formed of a nickel titanium alloy. The second wire is formed of a cobalt chromium alloy. The core wire includes one or more projections projecting from a joint surface between the first wire and the second wire toward a distal end side of the core wire in a longitudinal cross section including the joining surface. The one or more projections are formed of an alloy containing at least one element forming the nickel titanium alloy and at least one element forming the cobalt chromium alloy.

It is conventionally considered that, when different metal materials are joined via thermal history in which each of the metal materials are fused, an intermetallic compound is formed in a joint part between the materials and the joint part becomes brittle and joint strength is reduced. However, the inventors of the present application have conducted intensive studies and found that, when a core wire includes, in a joint part between a first wire and a second wire, one or more projections formed of an alloy containing at least one element forming a nickel titanium alloy being a forming material of the first wire and at least one element forming a cobalt chromium alloy being a forming material of the second wire, tensile strength of the joint part is improved. It is considered that this is because the projection partially intrudes into the nickel titanium alloy side, so that the contact area increases, and an anchoring effect occurs. Therefore, with this guide wire, it is possible to provide a guide wire including a core wire having excellent tensile strength while securing high flexibility in a distal end portion (portion formed by a first wire) of the core wire and high rigidity in a proximal end portion (portion formed by a second wire) of the core wire.

(2) The guide wire described above may have a configuration in which the one or more projections each have a length of greater than or equal to 2 μm along an axial direction of the core wire in the longitudinal cross section. With this configuration, the anchoring effect by the projection can be increased and tensile strength of the core wire can be effectively improved.

(3) The guide wire described above may have a configuration in which the alloy forming the one or more projections contains Ni, Ti, Co, and Cr. With this configuration, the joining property of the projection and the nickel titanium alloy and the cobalt chromium alloy can be further improved and tensile strength of the core wire can be further effectively improved.

(4) The guide wire described above may have a configuration in which a contour line of each of the one or more projections includes a portion extending toward an outer peripheral side of the projection and toward the distal end side of the core wire in the longitudinal cross section. With this configuration, the anchoring effect by the projection can be further increased and tensile strength of the core wire can be further effectively improved.

(5) The guide wire described above may have a configuration in which at least two of the one or more projections are not substantially continuous with each other due to the thickness of a layer of the alloy existing between the at least two projections, in the longitudinal cross section. With this configuration, since the two or more projections that are substantially not continuous from each other exist, tensile strength of the core wire can be further effectively improved.

(6) The guide wire described above may have a configuration in which the alloy forming the one or more projections is a material being more rigid than either the nickel titanium alloy or the cobalt chromium alloy. With this configuration, the anchoring effect can be further increased by the projection being relatively rigid and tensile strength of the core wire can be further effectively improved.

(7) A method for producing a guide wire disclosed in this specification includes steps of: controlling a roughness of an end surface of a first wire formed of a nickel titanium alloy such that an arithmetic mean height (Sa) is greater than or equal to 0.01 μm and less than or equal to 0.5 μm; and producing a core wire including the first wire and a second wire by abutting and joining the end surface of the first wire and an end surface of the second wire. According to this method for producing a guide wire, a projection can be formed in a joint part between the first wire and the second wire, and a guide wire including the core wire having excellent tensile strength can be produced. Note that the technology disclosed herein can be achieved in various aspects, such as guide wires, medical systems including a guide wire, and methods for producing those.

### B. Modification Example

The technology disclosed herein is not limited to the above embodiments, and can be modified in various forms without departing from the gist thereof. For example, the following modification examples are also possible.

In the embodiment described above, the core wire 10 includes the first wire 11 and the second wire 12. However, the core wire 10 may include another wire arranged in the proximal end side of the second wire 12.

In the embodiment described above, the core wire 10 includes a plurality of the projections 16. However, the core wire 10 may include a single projection 16.

In the embodiment described above, the joint part X1 between the first wire 11 and the second wire 12 of the core wire 10 is located in the proximal end side further than the proximal end of the coil body 20. However, the joint part X1 may be located in the distal end side further than the proximal end of the coil body 20.

The materials for the respective members in the embodiment described above are only examples, and may be modified variously. The method for producing the guide wire 100 in the embodiment described above is only an example and can be modified variously.

### REFERENCE SIGNS LIST

10: Core wire 11: First wire 12: Second wire 13: Small diameter portion 14: Tapered portion 15: Large diameter portion 16: Projection 18: Contour 18X: Folding back portion 20: Coil body 30: Distal end side joint part 40: Proximal end side joint part 100: Guide wire AX: Center axis S1: Joint surface X1: Joint part

1. A guide wire comprising:
a core wire including:
a first wire having a proximal end, the first wire being formed of a nickel titanium alloy;
a second wire having a distal end joined to the proximal end of the first wire, the second wire being formed of a cobalt chromium alloy; and
one or more projections projecting from a joint surface between the first wire and the second wire to a distal end side of the core wire in a longitudinal cross section including the joint surface,
the one or more projections being formed of an alloy containing at least one element forming the nickel titanium alloy and at least one element forming the cobalt chromium alloy.

2. The guide wire according to claim 1, wherein
the one or more projections each have a length of greater than or equal to 2 μm along an axial direction of the core wire in the longitudinal cross section.

3. The guide wire according to claim 1, wherein
the alloy forming the one or more projections contains Ni, Ti, Co, and Cr.

4. The guide wire according to claim 1, wherein
a contour line of each of the one or more projections in the longitudinal cross section includes a portion extending toward an outer peripheral side of the projection and toward the distal end side of the core wire.

5. The guide wire according to claim 1, wherein
at least two of the one or more projections are not substantially continuous with one another due to the thickness of a layer of the alloy existing between the at least two projections, in the longitudinal cross section.

6. The guide wire according to claim 1, wherein
the alloy forming the one or more projections is a material that is more rigid than either of the nickel titanium alloy or the cobalt chromium alloy.

7. A method for producing a guide wire comprising:
controlling a roughness of an end surface of a first wire formed of a nickel titanium alloy such that an arithmetic mean height (Sa) is greater than or equal to 0.01 μm and less than or equal to 0.5 μm; and
producing a core wire including the first wire and a second wire by abutting and joining the end surface of the first wire and an end surface of the second wire.
